(19) United States
(12) Patent Application Publication
(10) Pub. No.: US 2025/0325969 A1
(43) Pub. Date: Oct. 23, 2025

(54) METAL-ORGANIC FRAMEWORK COMPOUND WITH A PALLADIUM(II) SINGLE-SITE AND METHOD FOR PRODUCING 3-PHENYLPROPIONIC ACID USING THE SAME

(71) Applicant: National Taiwan University, Taipei City (TW)

(72) Inventors: Chia-Wen WU, Taipei City (TW); Ming-Rou WU, Taipei City (TW)

(21) Appl. No.: 18/757,666

(22) Filed: Jun. 28, 2024

(30) Foreign Application Priority Data
Apr. 23, 2024 (TW) 113115144

(51) Int. Cl. B01J 31/16; C07C 51/353

(52) U.S. Cl. CPC B01J 31/1691; C07C 51/353; B01J 2231/321; C07C 2601/14

(57) ABSTRACT

A metal-organic framework (MOF) compound with a palladium(II) single-site is represented by formula (I):

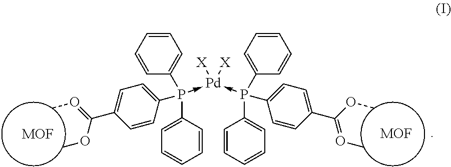

In formula (I), MOF represents an M-based MOF-808 (M-MOF-808), where M is a metal selected from the group consisting of zirconium (Zr), hafnium (Hf), and cerium (Ce), and X represents CH₃COO⁻. A method for producing 3-phenylpropionic acid using the aforesaid MOF compound is also provided.

### CROSS-REFERENCE TO RELATED APPLICATION

This application claims priority to Taiwanese Invention Patent Application No. 113115144, filed on Apr. 23, 2024, the entire disclosure of which is incorporated by reference herein.

### FIELD

The disclosure relates to a metal-organic framework (MOF) compound with a palladium(II) single-site, and a method for producing 3-phenylpropionic acid using the same.

### BACKGROUND

Palladium is often used as a catalyst in a hydroxycarbonylation reaction of an olefinic compound and a formic acid, and most palladium catalysts are homogeneous catalysts. Although the catalytic effect (e.g., conversion rate and yield) of homogeneous catalysts in a chemical reaction is generally better than that of heterogeneous catalysts, such homogeneous catalysts are difficult to be recycled and reused.

Even though the heterogeneous catalysts have the potential to be recycled, their metal active sites are often separated from supports (e.g., metal-organic frameworks (MOFs)) and dispersed in a solvent after the chemical reaction. Hence, the heterogeneous catalysts that contain palladium still remain difficult to be recycled.

### SUMMARY

Accordingly, in a first aspect, the present disclosure provides a metal-organic framework (MOF) compound with a palladium(II) single-site, which can alleviate at least one of the drawbacks of the prior art. The MOF compound is represented by formula (I):

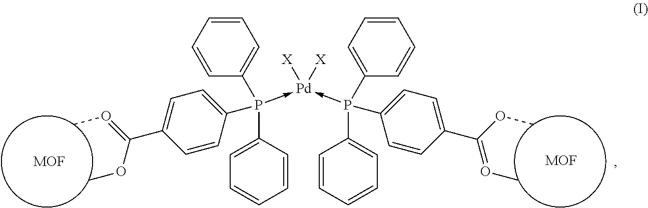

wherein
MOF represents an M-based MOF-808 (M-MOF-808), M being a metal selected from the group consisting of zirconium (Zr), hafnium (Hf), and cerium (Ce); and
X represents CH₃COO⁻.

In a second aspect, the present disclosure provides a method for producing 3-phenylpropionic acid, which can alleviate at least one of the drawbacks of the prior art. The method includes subjecting styrene and formic acid to a hydroxycarbonylation reaction in the presence of the aforesaid MOF compound.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a metal-organic framework (MOF) compound with a palladium(II) single-site. The MOF compound is represented by formula (I):

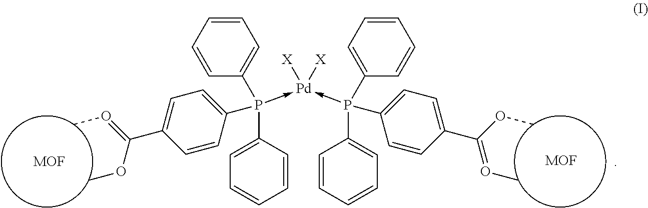

In the formula (I), MOF represents an M-based MOF-808 (M-MOF-808), and M is a metal selected from the group consisting of zirconium (Zr), hafnium (Hf), and cerium (Ce). In addition, X represents CH₃COO⁻.

In certain embodiments, M is Zr.

In certain embodiments, the MOF compound with the palladium(II) single-site has a specific surface area ranging from 300 m²/g to 350 m²/g. In an exemplary embodiment, the MOF compound with the palladium(II) single-site has the specific surface area of approximately 323 m²/g.

The present disclosure also provides a method for producing 3-phenylpropionic acid, which includes subjecting styrene and formic acid to a hydroxycarbonylation reaction in the presence of the aforesaid MOF compound with the palladium(II) single-site.

In certain embodiments, the hydroxycarbonylation reaction is carried out at a temperature ranging from 60° C. to 100° C. In an exemplary embodiment, the hydroxycarbonylation reaction is carried out at approximately 80° C.

In certain embodiments, a solvent used in the hydroxycarbonylation reaction is toluene.

In certain embodiments, in the hydroxycarbonylation reaction, a conversion rate of the styrene is greater than 99%. In an exemplary embodiment, the conversion rate of the styrene is approximately 100%.

In certain embodiments, in the hydroxycarbonylation reaction, a yield of the 3-phenylpropionic acid is greater than 45%. In an exemplary embodiment, the yield of the 3-phenylpropionic acid is approximately 49%.

The disclosure will be further described by way of the following example and comparative examples. However, it should be understood that the following examples are solely intended for the purpose of illustration and should not be construed as limiting the disclosure in practice.

### Example

[Synthesis of Metal-Organic Framework (MOF) Compound with Palladium(II) Single-Site]

First, 4.3 g of zirconium(IV) oxychloride octahydrate (ZrOCl₂˜8H₂O) and 0.9 g of 1,3,5-benzenetricarboxylic acid (H₃BTC) were added to a solvent mixture of 200 mL of dimethylformamide (DMF) and 200 mL of formic acid, followed by well mixing, so as to form a first solution. Subsequently, the first solution was placed in an oven having a temperature of 100° C. so as to allow a first reaction to proceed for 48 hours, followed by centrifugation, so as to obtain a first supernatant fraction and a first precipitate. After that, the first supernatant fraction was removed, and the first precipitate was sequentially rinsed with DMF, deionized water, and acetone, followed by lyophilization overnight, thereby obtaining Zr-MOF-808 (serving as an M-MOF-808).

Next, 2.5 g of the Zr-MOF-808 and 0.565 g of 4-(diphenylphosphine)benzoic acid were added to 250 mL of acetone (serving as a solvent), followed by well mixing, so as to form a second solution. Subsequently, the second solution was subjected to stirring to allow a second reaction to proceed for 24 hours, followed by centrifugation, so as to obtain a second supernatant fraction and a second precipitate. Thereafter, the second supernatant fraction was removed, and the second precipitate was rinsed with a fresh acetone, followed by lyophilization overnight, thereby obtaining PPh₃-Zr-MOF-808 (PPh₃ represents triphenylphosphine).

After that, 2.5 g of the PPh₃-Zr-MOF-808 and 0.1646 g of palladium(II) acetate (Pd(OAc)₂) were added to 170 mL of acetone (serving as another solvent), followed by well mixing, so as to form a third solution. Subsequently, the third solution was subjected to stirring to allow a third reaction to proceed overnight, followed by centrifugation, so as to obtain a third supernatant fraction and a third precipitate. Thereafter, the third supernatant fraction was removed, and the third precipitate was rinsed with another fresh acetone, followed by lyophilization overnight, thereby obtaining an MOF compound with a palladium(II) single-site of the Example.

The MOF compound with the palladium(II) single-site thus obtained was subjected to extended X-ray absorption fine structure (EXAFS) spectroscopy, so as to obtain an extended X-ray absorption fine structure (EXAFS) spectrum of such MOF compound. It could be confirmed from the EXAFS spectrum that the MOF compound with the palladium(II) single-site of the Example indeed contained Pd—O bonds and Pd—P bonds.

### [Measurement of Specific Surface Area]

The MOF compound with the palladium(II) single-site of the Example was subjected to nitrogen absorption-desorption isotherm analysis, followed by calculating a specific surface area by the Burnauer-Emmett-Teller (BET) method. The thus calculated specific surface area of the MOF compound with the palladium(II) single-site of the Example is 323.3 m²/g.

### [Preparation of 3-Phenylpropionic Acid]

First, 0.539 mL of styrene, 0.689 mL of formic acid, and 1.33 mL of acetic anhydride were added to 4.688 mL of toluene, followed by well mixing, so as to form a solution. Subsequently, the solution was placed in an autoclave, followed by conducting a hydroxycarbonylation reaction in the presence of 2.5 g of the MOF compound with the palladium(II) single-site of the Example (serving as a heterogeneous catalyst) at 80° C. for 18 hours. Next, the resultant mixture was subjected to centrifugation to obtain a precipitate and a supernatant. After that, reactants (the styrene and the formic acid) and product (3-phenylpropionic acid) in the supernatant were separated by gas chromatography, followed by determining a conversion rate of the styrene and a yield of the 3-phenylpropionic acid (calculated based on an initial amount of the styrene). The results are shown in Table 1 below.

### Comparative Example

The procedures and conditions in a method for preparing 3-phenylpropionic acid of Comparative Example were similar to those of the Example, except that the MOF compound with the palladium(II) single-site used in the Example was replaced with 0.1646 g of Pd(OAc)₂ and 0.4838 g of PPh₃ (both serving as a homogeneous catalyst). A conversion rate of styrene and a yield of 3-phenylpropionic acid were also determined as described in the Example. The results are shown in Table 1 below.

TABLE 1

| | Conversion | Yield of 3-phenyl- |
|---|---|---|
| | rate of styrene | propionic acid |
| Example | 100% | 48.8% |
| Comparative Example | 35% | 2% |

Referring to Table 1, the conversion rate of the styrene and the yield of the 3-phenylpropionic acid in the hydroxycarbonylation reaction of the Example were respectively much higher than the conversion rate of the styrene and the yield of the 3-phenylpropionic acid in the hydroxycarbonylation reaction of the Comparative Example, indicating that the MOF compound with the palladium(II) single-site of the Example has an excellent catalytic effect of converting the styrene into the 3-phenylpropionic acid in the hydroxycarbonylation reaction.

In summary, the MOF compound with the palladium(II) single-site of the present disclosure can be used as a heterogeneous catalyst, and has an excellent catalytic effect of converting styrene into 3-phenylpropionic acid in the hydroxycarbonylation reaction, and palladium active sites thereof does not separate from metal-organic frameworks (i.e., the M-based MOF-808 (M-MOF-808)) after the hydroxycarbonylation reaction, thereby facilitating recycling and reuse of such MOF compound, and hence achieving the purpose of the invention.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A metal-organic framework (MOF) compound with a palladium(II) single-site, the MOF compound being represented by formula (I): 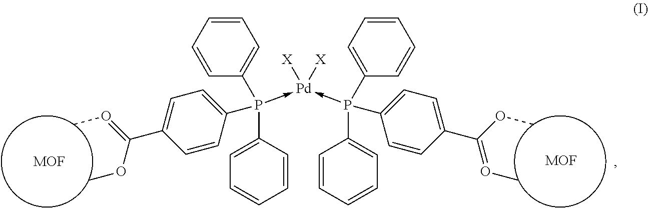
wherein
MOF represents an M-based MOF-808 (M-MOF-808), M being a metal selected from the group consisting of zirconium (Zr), hafnium (Hf), and cerium (Ce); and
X represents CH₃COO⁻.

2. The MOF compound as claimed in claim 1, wherein M is Zr.

3. The MOF compound as claimed in claim 1, which has a specific surface area ranging from 300 m²/g to 350 m²/g.

4. A method for producing 3-phenylpropionic acid, comprising subjecting styrene and formic acid to a hydroxycarbonylation reaction in the presence of an MOF compound as claimed in claim 1.

5. The method as claimed in claim 4, wherein the hydroxycarbonylation reaction is carried out at a temperature ranging from 60° C. to 100° C.

6. The method as claimed in claim 4, wherein a solvent used in the hydroxycarbonylation reaction is toluene.

7. The method as claimed in claim 4, wherein in the hydroxycarbonylation reaction, a conversion rate of the styrene is greater than 99%.

8. The method as claimed in claim 4, wherein in the hydroxycarbonylation reaction, a yield of the 3-phenylpropionic acid is greater than 45%.
